(19) United States
(12) Patent Application Publication
(10) Pub. No.: US 2005/0049283 A1
(43) Pub. Date: Mar. 3, 2005

(54) Compounds that inhibit factor xa activity

(76) Inventors: Robert Eckl, Munchen (DE); Silke Schabbert, Munchen (DE); Thilo Fuchs, Munchen (DE); Lutz Weber, Germering (DE); Christian Oefner, Freiburg (DE)

Correspondence Address: PALMER & DODGE, LLP;PAULA CAMPBELL EVANS, 111 HUNTINGTON AVENUE, BOSTON, MA, 02199, US

(21) Appl. No.: 10/501,932

(22) PCT Filed: Jan. 31, 2003
(86) PCT No.: PCT/EP03/01011

(30) Foreign Application Priority Data
Jan. 31, 2002 (DE) 1004072.9
Jan. 3, 2003 (DE) 10300049.6

(51) Int. Cl. A61K 31/4439; A61K 31/4245; C07D 413/02

(52) U.S. Cl. 514/341; 514/364; 546/269.1; 548/132

(57) ABSTRACT

The present invention related to compounds of formula (I): 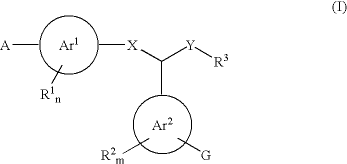 or pharmaceutically acceptable salts, hydrates or pharmaceutically acceptable formulations thereof. Those compounds can be used in inhibiting factor Xa and in the prevention and/or treatment of thromboembolic conditions.

The present invention relates to new compounds having an inhibitory action on blood clotting (so-called anticoagulants) and to their pharmacologically acceptable salts and solvates and hydrates, to pharmaceutical compositions comprising them as active ingredient, to processes for the preparation of such compounds, salts and compositions, and to the use thereof in the prevention and/or treatment of thromboembolic conditions. Those compounds, salts and compositions are very effective factor Xa inhibitors. The present invention relates also to pro-drugs, optically active forms, racemates and diastereoisomers of those compounds and salts.

Thromboembolic conditions are caused by an increased tendency to blood clotting in people with risk factors, such as, for example relatively major operations, prolonged immobilisation, fractures of the lower extremities, obesity, blood fat metabolism disorders, infections with gram-negative organisms, cancer and older age.

Venous thromboses may lead to the development of oedema or inflammation of the tissue drained by the affected vein. Thrombosis of a deeper vein (so-called deep vein thrombosis) may lead to serious complications, such as, for example, pulmonary embolism. Arterial thrombosis may lead to ischaemic necrosis of the tissue supplied by the affected artery, such as, for example, to myocardial infarct in the case of an affected:coronary artery. Other thromboembolic conditions are, for example, arterio-sclerosis, apoplexy (stroke), angina pectoris, intermittent claudication.

Under normal physiological conditions, natural blood clotting protects against major blood loss from a damaged blood vessel. During blood clotting, liquid blood is converted into a blood clot, a gelatinous mass which seals injured blood vessels by forming a plug. In that process, soluble fibrinogen present in the plasma is converted into the fibrous-gelatinous clotting substance fibrin in a multi-stage process, the so-called coagulation cascade.

A distinction is made between two different pathways of coagulation activation. The intrinsic coagulation pathway is initiated when blood comes into contact with non-physiological surfaces. The extrinsic coagulation pathway is initiated by injury to blood vessels. Both coagulation pathways join in a common pathway in which the coagulation factor X, a serine protease, is converted into its active form (factor Xa). Factor Xa, together with factor Va and Ca²⁺ in the so-called prothrombinase complex, causes prothrombin to be converted into thrombin which in turn, by cleaving peptides from fibrinogen, releases fibrin monomers, which are capable of coagulating to form fibrin fibres. Finally, factor XIII brings about cross-linking and thus stabilisation of the fibrin fibres.

Anticoagulants are used both for the prevention and for the treatment of thromboembolic conditions. As far as anticoagulants in the narrower sense are concerned, a distinction is made between heparin, which is immediately effective and which directly inhibits certain blood clotting factors, and vitamin K antagonists (for example, coumarin derivatives). The latter inhibit the production in the liver of certain clotting factors which is dependent on the presence of vitamin K, and begin to take effect only slowly. Other anticoagulant agents are the fibrinolytics, which bring about direct or indirect activation of the fibrinolytic system, and thrombocyte aggregation inhibitors, such as, for example, acetylsalicylic acid. A more seldom used method is reduction of the fibrinogen level in the blood by the enzyme ancrod. The object of using anticoagulant agents is to prevent the development of a blood clot that could close a vessel or also to dissolve it again once it has formed.

The above-mentioned anticoagulants in the narrower sense, that is to say heparin and vitamin K antagonists, have disadvantages. In the case of heparin, a distinction is made between unfractionated heparin (UFH) and low-molecular-weight heparin (LMWH). A disadvantage with UFH is the fact that it generally has to be administered intravenously, has a varying anticoagulant effect and therefore necessitates frequent monitoring of the patient and adaptation of the dosage. Although LMWH can be used subcutaneously in a constant, unmonitored dosage, its effect, compared to that of UFH, is greatly reduced because of its short chain length.

The vitamin K antagonists such as, for example, warfarin exhibit degrees of activity that differ from patient to patient, presumably owing to genetic factors. In addition to the slow onset of action mentioned above, this is associated with the disadvantage that patients have to be monitored and individual adaptation of the dosage is required.

Other known anticoagulants belong to the group of the thrombin inhibitors. Current overviews of relevant research activity in that field can be found, for example, in Jules A. Shafer, Current Opinion in Chemical Biology, 1988, 2: 458-485, Joseph P. Vacca, Current Opinion in Chemical Biology, 2000, 4: 394-400 and also in Fahad Al-Obeidi and James A. Ostrem, DDT, Vol. 3, No. 5, May 1998: 223-231.

A crucial disadvantage of thrombin inhibitors is that, in order to obtain the desired effect, it is necessary to suppress thrombin activity in vivo to such a great extent that the tendency to haemorrhage may increase, which makes dosage difficult.

In contrast, factor Xa inhibitors cause suppression of the new formation of thrombin from prothrombin, whereas they do not impair existing thrombin activity which is necessary for primary haemostasis.

The spectra of action and side-effects of some of those factor Xa inhibitors have not yet been fully investigated.

An object of the present invention was to provide new compounds having useful properties, especially an anticoagulating action.

More precisely, the object was to provide new factor Xa inhibitors having improved efficacy, reduced side-effects and/or increased selectivity. In addition, suitable pharmaceutical compositions were to be provided. Those compounds and compositions were to be administrable preferably parenterally or orally, especially orally.

A further object of the present invention was to provide a process for the preparation of those new compounds.

Those new compounds were furthermore to be suitable for use in the prevention and/or treatment of thromboembolic conditions.

The present invention describes anticoagulant compounds, their pharmacologically acceptable salts and solvates and hydrates and formulations that have a high activity and selectivity and can be administered especially orally. The present invention further relates to pro-drugs, optically active forms, racemates and diastereoisomers of those compounds and salts. The said compounds and salts may also themselves be pro-drugs, which are activated only by metabolisation. Pharmaceutical compositions comprising the said compounds or salts etc. as active ingredient are also described.

The present invention relates to a compound of the general formula (I): 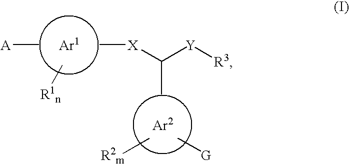
wherein
A is a hydrogen atom; a group of formula —NHC(═NR⁴)NH₂ or —C(═NR⁴)NH₂, wherein R⁴ is a hydrogen atom, a heteroalkyl, heteroaralkyl, heterocycloalkyl, heteroalkylcycloalkyl, hydroxy or alkyloxy group, or R⁴, together with one of the radicals R¹, is part of a 5- or 6-membered heteroaryl or heterocycloalkyl ring; or A has one of the following structures: 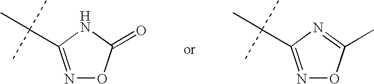
   Ar¹ is an aryl, aralkyl, heteroaryl or heteroaralkyl group,
Ar² is an aryl, aralkyl, heteroaryl or heteroaralkyl group,
the radicals R¹ are, each independently of any other(s), a hydrogen atom, a hydroxy group, a C₁—, C₂—, C₃— or C₄- alkyloxy group, an amino group, a C₁—, C₂—, C₃— or C₄-alkylamino group, a C₁—, C₂—, C₃— or C₄-dialkylamino group, a cyano group or a halogen atom;
the radicals R², each independently of any other(s), are a hydrogen atom, a hydroxy group, a C₁—, C₂—, C₃— or C₄-alkyloxy group, an amino group, a C₁—, C₂—, C₃— or C₄-group, a C₁—, C₂—, C₃— or C₄-dialkylamino group,alkylamino a cyano group or a halogen atom;
R³ is an alkyl, alkenyl, alkynyl, heteroalkyl, aryl, heteroaryl, cycloalkyl, alkylcycloalkyl, heteroalkylcyclo-alkyl, heterocycloalkyl, aralkyl or heteroaralkyl radical;
G is a glycosyl group;
X is a group of formula NR⁵, O, CONR⁵, NR⁵CO, CH₂NR⁵, S, SO, SO₂, SO₂NH, NHSO₂, PO₂NH, NHPO₂, CH₂, CHMe or CO, wherein R⁵ is a hydrogen atom, a C₁—, C₂—, C₃— or C₄-alkyl group, a C₁—, C₂—, C₃— or C₄-heteroalkyl group, a C₇—, C₈—, C₉—, C₁₀—, C₁₁— or C₁₂-aralkyl group, or a C₆—, C₇—, C₈—, C₉—, C₁₀—, C₁₁— or C₁₂-heteroaralkyl group;
Y is a group of formula CONR⁶, COCONR⁶, NR⁶, O, NR⁶CO, S, SO, SO₂, SO₂NH, NHSO₂, PO₂NH, NHPO₂, CH₂, CHMe or CO, wherein R⁶ is a hydrogen atom, a C₁—, C₂—, C₃— or C₄-alkyl group, a C₁—, C₂—, C₃— or C₄-heteroalkyl group or a C₇—, C₈—, C₉—, C₁₀—, C₁₁— or C₁₂-aralkyl group;
n is 0, 1, 2, 3 or 4, and
m is 0, 1, 2, 3 or 4,
or a pharmacologically acceptable salt, solvate, hydrate or pharmacologically acceptable formulation thereof.

The following definitions relate to the entire description and, especially, to the claims:

The expression alkyl refers to a saturated, straight-chain or branched hydrocarbon group having, for example, from 1 to 20 carbon atoms, preferably from 1 to 12 carbon atoms, especially 1, 2, 3, 4, 5 or 6 carbon atoms, for example a methyl, ethyl, propyl, isopropyl, isobutyl, tert-butyl, n-hexyl, 2,2-dimethylbutyl or n-octyl group.

The expression alkenyl refers to straight-chain or branched hydrocarbon groups containing at least one double bond and having from 2 to 20 carbon atoms, preferably from 2 to 12 carbon atoms, especially 2, 3, 4, 5, or 6 carbon atoms, for example an ethenyl, allyl, isoprenyl or hex-2-enyl group. They preferably have one or two (especially one) double bond(s).

The expression alkynyl refers to straight-chain or branched hydrocarbon groups containing at least one triple bond and having from 2 to 20 carbon atoms, preferably from 2 to 12 carbon atoms, especially 2, 3, 4, 5, or 6 carbon atoms, for example an ethynyl or propargyl group. They preferably have one or two (especially one) triple bond(s).

Furthermore, the terms alkyl, alkenyl and alkynyl refer to groups in which one, two, three or more hydrogen atoms have been replaced by a halogen atom (preferably F or Cl) such as, for example, a 2,2,2-trichloroethyl or trifluoromethyl group.

The expression heteroalkyl refers to an alkyl, alkenyl or alkynyl group in which one or more (preferably 1, 2 or 3) carbon atoms have been replaced, each-independently of any other(s), by an oxygen, nitrogen, phosphorus, boron, selenium, silicon and/or sulphur atom (preferably oxygen, sulphur or nitrogen). The expression heteroalkyl furthermore refers to a carboxylic acid or to a group derived from a carboxylic acid such as, for example, acyl, acylalkyl, alkoxycarbonyl, acyloxy, acyloxyalkyl, carboxyalkylamide or alkoxycarbonyloxy.

Examples of heteroalkyl groups are groups of formulae R^{a}—O—Y^{a}—, R^{a}—S—Y^{a}—, R^{a}—N(R^{b})—Y^{a}—, R^{a}—CO—Y^{a}—, R^{a}—O—CO—Y^{a}—, R^{a}—CO—O—Y^{a}—, R^{a}—CO—N(R^{b})—Y^{a}—, R^{a}—N(R^{b})—CO—Y^{a}—, R^{a}—O—CO—N(R^{b})—Y^{a}—, R^{a}—N(R^{b})—CO—O—Y^{a}—, R^{a}—N(R^{b})—CO—N(R^{c})—Y^{a}—, R^{a}—O—OC—O—Y^{a}—, R^{a}—N(R^{b})—C(═NR^{d})—N(R^{c})—Y^{a}, R^{a}—CS—Y^{a}—, R^{a}—O—CS—Y^{a}—, R^{a}—CS—O—Y^{a}—, R^{a}—CS—N(R^{b})—Y^{a}—, R^{a}—N(R^{b})—CS—Y^{a}—, R^{a}—O—CS—N(R^{b})—Y^{a}—, R^{a}—N(R^{b})—CS—O—Y^{a}—, R^{a}—N(R^{b})—CS—N(R^{c})—Y^{a}—, R^{a}—O—CS—O—Y^{a}—, R^{a}—S—CO—Y^{a}—, R^{a}—CO—S—Y^{a}, R^{a}—S—CO—N(R^{b})—Y^{a}—, R^{a}—N(R^{b})—CO—S—Y^{a}—, R^{a}—S—CO—O—Y^{a}—, R^{a}—CO—S—Y^{a}—, R^{a}—S—CO—S—Y^{a}—, R^{a}—S—CS—Y^{a}—, R^{a}—CS—S—Y^{a}—, R^{a}—S—CS—N(R^{b})—Y^{a}—, R^{a}—N(R^{b})—CS—S—Y^{a}—, R^{a}—S—CS—O—Y^{a}—, R^{a}—O—CS—S—Y^{a}—, R^{a} being a hydrogen atom, a C₁—, C₂—, C₃—, C₄—, C₅— or C₆-alkyl, C₂—, C₃—, C₄—, C₅— or C₆-alkenyl or C₂—, C₃—, C₄—, C₅— or C₆-alkynyl group; R^{b} being a hydrogen atom, a C₁—, C₂—, C₃—, C₄—, C₅— or C₆-alkyl, C₂—, C₃—, C₄—, C₅— or C₆-alkenyl or C₂—, C₃—, C₄—, C₅— or C₆-alkynyl group; R^{c} being a hydrogen atom, a C₁—, C₂—, C₃—, C₄—, C₅— or C₆-alkyl, C₂—, C₃—, C₄—, C₅— or C₆-alkenyl or C₂—, C₃—, C₄—, C₅— or C₆-alkynyl group; R^{d} being a hydrogen atom, a C₁—, C₂—, C₃—, C₄—, C₅— or C₆-alkyl, C₂—, C₃—, C₄—, C₅— or C₆-alkenyl or C₂—, C₃—, C₄—, C₅— or C₆-alkynyl group and Y^{a} being a bond, a C₁—, C₂—, C₃—, C₄—, C₅— or C₆-alkylene, C₂—, C₃—, C₄—, C₅— or C₆-alkenylene or C₂—, C₃—, C₄—, C₅— or C₆-alkynylene group, each heteroalkyl group containing at least one carbon atom and it being possible for one, two, three or more hydrogen atoms to have been replaced by halogen atoms (especially fluorine or chlorine atoms). Specific examples of heteroalkyl groups are methoxy, trifluoromethoxy, ethoxy, n-propyloxy, isopropyloxy, tert-butyloxy, methoxymethyl, ethoxymethyl, methoxyethyl, methylamino, ethylamino, dimethylamino, diethylamino, isopropylethylamino, methylaminomethyl, ethylaminomethyl, diisopropylaminoethyl, enol ether, dimethylaminomethyl, dimethylaminoethyl, acetyl, propionyl, butyryloxy, acetyloxy, methoxycarbonyl, ethoxycarbonyl, N-ethyl-N-methylcarbamoyl and N-methylcarbamoyl. Further examples of heteroalkyl groups are nitrile, isonitrile, cyanate, thiocyanate, isocyanate, isothiocyanate, carbonyl and alkylnitrile groups.

The expression cycloalkyl refers to a saturated or partially unsaturated (for example, cycloalkenyl, cycloalkynyl) cyclic group having one or more rings (preferably 1 or 2, especially 1) and containing a total of from 3 to 14 ring carbon atoms, preferably from 3 to 10 (especially 3, 4, 5, 6 or 7) ring carbon atoms. The expression cycloalkyl refers furthermore to corresponding groups in which one or more hydrogen atoms have been replaced, each independently of any other(s), by fluorine, chlorine, bromine or iodine atoms or by OH, ═O, SH, ═S, NH₂, ═NH or NO₂ groups, that is to say, for example, cyclic ketones such as, for example, cyclohexanone, 2-cyclohexenone or cyclopentanone. Further specific examples of cycloalkyl groups are a cyclopropyl, cyclobutyl, cyclopentyl, spiro[4,5]decanyl, norbornyl, cyclohexyl, cyclopentenyl, cyclohexadienyl, decalinyl, cubanyl, bicyclo[4.3.0]nonyl, 1,2,3,4-tetrahydronaphthyl, cyclo-pentylcyclohexyl, fluorocyclohexyl or cyclohex-2-enyl group.

The expression heterocycloalkyl refers to a cycloalkyl group as defined above (for example, saturated or mono- or poly-unsaturated cycloalkyl groups such as cycloalkenyl groups) in which one or more (preferably 1, 2 or 3) ring carbon atoms have been replaced, each independently of any other(s), by an oxygen, nitrogen, silicon, selenium, phosphorus or sulphur atom (preferably oxygen, sulphur or nitrogen). A heterocycloalkyl group has preferably 1 or 2 (especially 1) ring(s) containing from 3 to 10 (especially 3, 4, 5, 6 or 7) ring atoms. The expression heterocycloalkyl refers furthermore to corresponding groups in which one or more hydrogen atoms have been replaced, each independently of any other(s), by fluorine, chlorine, bromine or iodine atoms or by OH, ═O, SH, ═S, NH₂, ═NH or NO₂ groups. Examples are a piperidyl, morpholinyl, urotropinyl, pyrrolidinyl, tetrahydrothiophenyl, tetra-hydropyranyl, tetrahydrofuryl, oxacyclopropyl, azacyclo-propyl or 2-pyrazolinyl group and also lactams, lactones, cyclic imides and cyclic anhydrides.

The expression alkylcycloalkyl refers to groups containing both cycloalkyl and also alkyl, alkenyl or alkynyl groups in accordance with the above definitions. An alkylcyloalkyl group contains preferably one or two (especially one) cycloalkyl group(s), each of which contains from 3 to 10 (especially 3, 4, 5, 6 or 7) ring carbon atoms, and one or two alkyl, alkenyl or alkynyl groups containing 1 or 2 to 6 carbon atoms.

Examples of such compounds are:
alkylcycloalkyl, alkyldicycloalkyl, dialkylcycloalkyl. alkylcycloalkenyl, alkyldicycloalkenyl, dialkylcycloalkenyl, alkenylcycloalkyl, alkenyldicycloalkyl, dialkyldicycloalkyl, alkenylcycloalkenyl, alkenyldicycloalkenyl, dialkyldicycloalkyl, dialkenylcycloalkyl, dialkenylcycloalkenyl, alkynylcycloalkyl, alkynyldicycloalkyl, dialkenyldicycloalkyl, alkynylcycloalkenyl, alkynyldicycloalkenyl, dialkenyldicycloalkenyl.

The expression heteroalkylcycloalkyl refers to alkylcycloalkyl groups as defined above in which one or more (preferably 1, 2 or 3) carbon atoms have been replaced, each independently of any other(s), by an oxygen, nitrogen, silicon, selenium, phosphorus or sulphur atom (preferably oxygen, sulphur or nitrogen). A heteroalkylcycloalkyl group contains preferably 1 or 2 (especially one) ring(s) each containing from 3 to 10 (especially 3, 4, 5, 6 or 7) ring atoms and one or two alkyl, alkenyl, alkynyl or heteroalkyl groups each containing 1 or 2 to 6 carbon atoms. Examples of such groups, which may be substituted by alkyl, alkenyl, alkynyl and/or heteroalkyl groups, are:
heteroalkylcycloalkyl, heteroalkyldicycloalkyl, diheteroalkylcycloalkyl, heteroalkylcycloalkenyl, heteroalkyldicycloalkenyl, diheteroalkylcycloalkenyl, heteroalkenylcycloalkyl, heteroalkenyldicycloalkyl, diheteroalkyldicycloalkyl, heteroalkenylcycloalkenyl, heteroalkenyldicycloalkenyl, diheteroalkyldicycloalkyl, diheteroalkenylcycloalkyl, diheteroalkenylcycloalkenyl, heteroalkynylcycloalkyl, heteroalkynyldicycloalkyl, diheteroalkenyldicycloalkyl, heteroalkynylcycloalkenyl, heteroalkynyldicycloalkenyl, diheteroalkenyldicycloalkenyl, alkylheterocycloalkyl, alkyldiheterocycloalkyl, dialkylheterocycloalkyl, alkylheterocycloalkenyl, alkyldiheterocycloalkenyl, dialkylheterocycloalkenyl, alkenylheterocycloalkyl, alkenyldiheterocycloalkyl, dialkyldiheterocycloalkyl, alkenylheterocycloalkenyl, alkenyldiheterocycloalkenyl, dialkyldiheterocycloalkyl, dialkenylheterocycloalkyl, dialkenylheterocycloalkenyl, alkynylheterocycloalkyl, alkynyldiheterocycloalkyl, dialkenyldiheterocycloalkyl, alkynylheterocycloalkenyl, alkynyldiheterocycloalkenyl, dialkenyldiheterocycloalkenyl, heteroalkylheterocycloalkyl, heteroalkyldiheterocycloalkyl, diheteroalkylheterocyclo-alkyl, heteroalkylheterocycloalkenyl, heteroalkyldiheterocycloalkenyl, diheteroalkylheterocycloalkenyl, heteroalkenylheterocycloalkyl, heteroalkenyldiheterocyclo-alkyl, diheteroalkyldiheterocycloalkyl, heteroalkenylheterocycloalkenyl, heteroalkenyldiheterocycloalkenyl, diheteroalkyldiheterocycloalkyl, diheteroalkenylheterocycloalkyl, diheteroalkenylheterocycloalkenyl, hetero-alkynylheterocycloalkyl, heteroalkynyldiheterocycloalkyl, diheteroalkenyldiheterocycloalkyl, heteroalkynylheterocycloalkenyl, heteroalkynyldiheterocycloalkenyl, diheteroalkenyldiheterocycloalkenyl.

Special preference is given to:
alkylheterocycloalkyl, alkylheterocycloalkenyl, alkenylheterocycloalkyl, alkynylheterocycloalkyl, heteroalkylcycloalkyl, heteroalkylheterocycloalkyl and heteroalkylheterocylcloalkenyl, the cyclic groups being saturated or mono-, di- or tri-unsaturated.

The expression aryl or Ar refers to an aromatic group which has one or more rings, preferably one ring, containing from 6 to 14 ring carbon atoms, preferably from 6 to 10 (especially 6) ring carbon atoms. The expression aryl (or Ar) refers furthermore to corresponding groups in which one or more hydrogen atoms have been replaced, each independently of any other(s), by fluorine, chlorine, bromine or iodine atoms or by OH, SH, NH₂ or NO₂ groups. Examples are a phenyl, naphthyl, biphenyl, 2-fluorophenyl, anilinyl, 3-nitrophenyl or 4-hydroxyphenyl group.

The expression heteroaryl refers to an aromatic group which has one or more rings, preferably one ring, containing from 5 to 14 ring atoms, preferably from 5, 6, 7, 8, 9 or 10 (especially 5 or 6) ring atoms, one or more (preferably 1, 2, 3 or 4) ring atoms having been replaced by oxygen, nitrogen, phosphorus or sulphur ring atoms (preferably O, S or N). The expression heteroaryl refers furthermore to corresponding groups in which one or more hydrogen atoms have been replaced, each independently of any other(s), by fluorine, chlorine, bromine or iodine atoms or by OH, SH, NH₂ or NO₂ groups. Examples are 4-pyridyl, 2-imidazolyl, 3-phenylpyrrolyl, thiazolyl, oxazolyl, triazolyl, tetrazolyl, isoxazolyl, indazolyl, indolyl (for example, 6-indolyl), benzimidazolyl, pyridazinyl, quinolyl, purinyl, carbazolyl, acridinyl, pyrimidyl, 2,3-bifuryl, 3-pyrazolyl and isoquinolyl groups.

The expression aralkyl refers to groups containing both aryl and also alkyl, alkenyl, alkynyl and/or cycloalkyl groups in accordance with the above definitions such as, for example, arylalkyl, arylalkenyl, arylalkynyl, arylcycloalkyl, arylcycloalkenyl, arylcycloalkynyl, alkylarylcycloalkyl, alkylarylcycloalkenyl, alkylarylcycloalkynyl, alkylarylalkyl, alkylarylalkenyl, alkylarylalkynyl, alkenylarylalkenyl, alkynylarylalkenyl, alkynylarylalkynyl and arylalkylcycloalkyl groups. Specific examples of aralkyls are toluene, trityl, xylene, mesitylene, styrene, benzyl chloride, o-fluorotoluene, 1H-indene, 1,2,3,4-tetra-hydronaphthyl, dihydronaphthalene, indanone, phenylcyclopentyl, cumene, cyclohexylphenyl, fluorene and indan. An aralkyl group preferably comprises an aromatic ring system (1 or 2 rings) containing from 6 to 10 carbon atoms (for example, phenyl or naphthyl) and one or two alkyl, alkenyl and/or alkynyl groups each containing 1 or 2 to 6 carbon atoms and/or a cycloalkyl group containing 5 or 6 ring carbon atoms.

The expression heteroaralkyl refers to an aralkyl group as defined above in which one or more (preferably 1, 2, 3, or 4) carbon atoms have been replaced, each independently of any other(s), by an oxygen, nitrogen, silicon, selenium, phosphorus, boron or sulphur atom (preferably oxygen, sulphur or nitrogen), that is to say to groups containing both aryl or heteroaryl and also alkyl, alkenyl or alkynyl and/or heteroalkyl and/or cycloalkyl or cycloalkenyl, and/or heterocycloalkyl or heterocycloalkenyl groups in accordance with the above definitions. A heteroaralkyl group preferably contains one or two aromatic ring systems (each comprising 1 or 2 rings) each containing 5, 6, 7, 8, 9 or 10 ring carbon atoms and one or two alkyl, alkenyl and/or alkynyl groups containing 1 or 2, 3, 4, 5 or 6 carbon atoms and/or a cycloalkyl group containing 5 or 6 ring carbon atoms, 1, 2, 3 or 4 or those carbon atoms and/or ring carbon atoms having been replaced, each independently of any other(s), by oxygen, sulphur or nitrogen atoms.

Examples are aryl-heteroalkyl, aryl-heterocycloalkyl, aryl-heterocycloalkenyl, aryl-alkyl-heterocycloalkyl, aryl-alkenyl-heterocycloalkyl, aryl-alkynyl-heterocycloalkyl, aryl-alkyl-heterocycloalkenyl, aryl-heteroalkyl-heterocycloalkyl, heteroaryl-alkyl, heteroaryl-alkenyl, hetero-aryl-alkynyl, heteroaryl-heteroalkyl, heteroaryl-cycloalkyl, heteroaryl-cycloalkenyl, heteroaryl-heterocycloalkyl, heteroaryl-heterocycloalkenyl, heteroaryl-alkyl-cycloalkyl, heteroaryl-heteroalkyl-cycloalkyl, heteroaryl-alkyl-heterocycloalkenyl, heteroaryl-heteroalkyl-cycloalkenyl and heteroaryl-heteroalkyl-heterocycloalkyl groups, the cyclic groups being saturated or mono-, di- or tri-unsaturated. Specific examples are a tetrahydro-isoquinolyl, benzoyl, 2- or 3-ethyl-indolyl, 4-methyl-pyridino, 2-, 3- or 4-methoxyphenyl, 4-ethoxyphenyl, 2-, 3- or 4-carboxyphenylalkyl group.

The expressions cycloalkyl, aryl, heterocycloalkyl, alkylcycloalkyl, heteroalkylcycloalkyl, heteroaryl, aralkyl and heteroaralkyl refer to groups in which one or more hydrogen atoms have been replaced, each independently of any other(s), by fluorine, chlorine, bromine or iodine atoms or by OH, ═O, SH, ═S, NH₂, ═NH or NO₂ groups (the ═O, ═S and ═NH groups in each case replacing two hydrogen atoms).

The expression “optionally substituted” refers to groups in which one, two or more hydrogen atoms have been replaced by fluorine, chlorine, bromine or iodine atoms or by OH, ═O, SH, ═S, SO₂NH₂, NH₂, ═NH or NO₂ groups. The expression refers furthermore to groups in which one, two or more hydrogen atoms have been replaced, each independently of any other(s), by unsubstituted C₁-C₆alkyl, C₂-C₆alkenyl, C₂-C₆alkynyl, C₁-C₆heteroalkyl, C₃-C₁₀cycloalkyl, C₂-C₉hetero-cycloalkyl, C₆-C₁₀aryl, C₁-C₉heteroaryl, C₇-C₁₂aralkyl or C₂-C₁₁heteroaralkyl groups.

In the present Application, the expression glycosyl group or glycosyl radical refers to a saccharide (mono- or oligo-saccharide, including amino sugars and N-acetylamino sugars) bonded by way of an α- or β-O—, —S—, —N— or —C-glycosidic bond (preferably an O-glycosidic bond), wherein the OH groups may optionally be protected by acetyl or benzoyl groups, especially a monosaccharide (for example, glucose, galactose, fructose, fucose, ribose, glucosamine, N-acetylglucosamine, galactosamine, N-acetylgalactosamine or mannose), preferably β-D-glucose.

Preference is given to compounds of the general formula (I) wherein Y is a group of formula CONR⁶ and R³ is not a group of formula —CHR⁷—CO—NR⁸R⁹, R⁷, R⁸ and R⁹ being, each independently of the others, a hydrogen atom, an alkyl, alkenyl, alkynyl, heteroalkyl, heteroaralkyl, heteroaryl, aralkyl, cycloalkyl, heterocycloalkyl, alkylcycloalkyl, heteroalkylcycloalkyl or aryl group or R⁸ and R⁹ together being part of a heterocycloalkyl or heteroaryl ring system.

Preference is furthermore given to compounds of the general formula (I) wherein Y is a group of formula CO and R³ is not a group of formula —NR¹⁰—CHR⁷—CO—NR⁸R⁹, R⁷, R , R⁹ and R¹⁰ being, each independently of the others, a hydrogen atom, an alkyl, alkenyl, alkynyl, heteroalkyl, heteroaralkyl, heteroaryl, alkylcycloalkyl, heteroalkyl-cycloalkyl, aralkyl, cycloalkyl, heterocycloalkyl or aryl group or R⁸ and R⁹ and/or R⁷ and R¹⁰ together being part of a heterocycloalkyl or heteroaryl ring system. Excluded, in particular, are compounds wherein the radical —Y—R³ has the following structure:
—C(═O)—NR—CR′R″—C(═O)—N(R′″)R^{IV}, the radicals R, R′, R″, R′″ and R^{IV} being defined as desired or denoting any desired chemical radicals.

Preference is also given to Ar¹ not being a phenyl ring to which A and X are bonded in positions para to one another when A is a group of formula —C(═NR⁴)NH₂ (especially ‘3C(═NH)NH₂).

Preference is furthermore given to A being a group of formula —C(═NR⁴)NH₂.

Preference is moreover given to A being a hydrogen atom.

Preference is also given to R⁴ being a hydrogen atom, a hydroxy or C₁—, C₂—, C₃— or C₄-alkyloxy group; special preference is given to R⁴ being a hydrogen atom.

Preference is furthermore given to Ar¹ being a phenyl group or a heteroaryl group containing 5, 6, 7, 8, 9 or 10 ring atoms and 1, 2, 3 or 4 hetero atoms which are selected from O, S and N; special preference is given to Ar¹ being a phenyl group, especially a phenyl group to which the groups A and X are bonded in positions meta to one another.

Preference is moreover given to Ar² being a phenyl group or a heteroaryl group containing 5 or 6 ring atoms and 1, 2 or 3 hetero atoms selected from O, S and N; special preference is given to Ar² being a phenyl group.

Preference is also given to X being a group of formula NH, NMe or NAc; special preference is given to X being an NH group.

Preference is moreover given to n being 0, 1 or 2, especially 0 or 1.

Preference is furthermore given to R¹ being a hydroxy group which, when Ar¹ is phenyl, is especially bonded in the position para to A.

Preference is also given to m being 0 or 1, the radicals R² and G preferably being in positions ortho to one another; special preference is given to m being 0.

Preference is furthermore given to Y being a group of formula CONH.

Preference is moreover given to R³ being a group of formula —U—V—W, wherein U is an optionally substituted arylene group containing 6 - 10 or 12 ring carbon atoms or an optionally substituted heteroarylene group containing 5, 6, 7, 8, 9 or 10 ring carbon atoms and 1, 2, 3 or 4 (preferably 1 or 2) hetero: atoms selected from O, S and N; V is a bond, an oxygen atom, a sulphur atom, a group of formula NR¹¹ (R¹¹ being a hydrogen atom, a C₁—, C₂—, C₃— or C₄-alkyl group, a C₁—, C₂—, C₃— or C₄-heteroalkyl group, a C₇—, C₈—, C₉—, C₁₀—, C₁₁— or C₁₂-aralkyl group or a C₆—, C₇—, C₈—, C₉—, C₁₀—, C₁₁— or C₁₂-heteroaralkyl group) , CO, SO, SO₂ or SO₂NH, and W is a hydrogen atom, an alkyl, alkenyl, alkynyl, heteroalkyl, aryl, heteroaryl, cycloalkyl, alkylcycloalkyl, heteroalkylcycloalkyl, heterocycloalkyl, aralkyl or heteroaralkyl radical.

Special preference is given to U being an optionally substituted phenylene group, especially a para-phenylene group.

Special preference is moreover given to V being a bond or a carbonyl group.

Special preference is also given to W being a C₁—, C₂—, C₃— or C₄-alkyl group, a C₁—, C₂—, C₃— or C₄-heteroalkyl group containing one or two O, N or S atoms, an optionally substituted phenyl group, an optionally substituted C₃—, C₄—, C₅—, C₆— or C₇-cycloalkyl group, an optionally substituted heterocycloalkyl group containing 3-7 (preferably 5 or 6) ring carbon atoms and 1, 2 or 3 ring hetero atoms (selected, each independently of any other(s), from O, S and N) or an optionally substituted heteroaryl group containing 5 or 6 ring carbon atoms and 1, 2, 3 or 4 ring hetero atoms selected from O, S and N.

Preference is given to compounds of the general formula I wherein Y is a group of formula CONR⁶ and R³ is not a group of formula —CHR⁷—CO—NR⁸R⁹ , R⁷, R⁸ and R⁹ being as defined for R⁵, R⁶ and R⁷ in the PCT Application PCT/EP 02/01934 (WO 02/068390) of the company Morphochem AG of 22nd Feb. 2002 or R⁷, R⁸ and R⁹ being as defined for R⁵, R⁶ and R⁷ in the PCT Application PCT/EP 01/09753 (WO 02/16312) of the company Morphochem AG of 23rd Aug. 2001.

Special preference is given to compounds of formula (II): 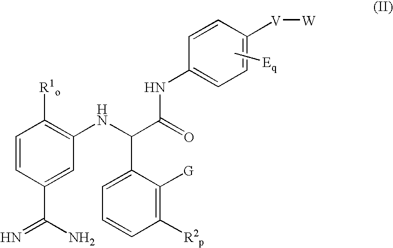
wherein E is a hydrogen, fluorine, chlorine or bromine atom and the radicals R¹, R², G, V and W are as defined hereinbefore, o is 0 or 1, p is 0 or 1, and q is 0, 1 or 2. Special preference is given to R¹ being a hydroxy group, R² being a methoxy or ethoxy group, G being a β-D-glucosyloxy group and V being a bond or a carbonyl group (C═O).

Special preference is given to W being a cyclic group of formula —N(CH₂CH₂)₂Q wherein Q is an oxygen atom or a group of formula NR¹², R¹² being a hydrogen atom, a C₁—, C₂—, C₃— or C₄-alkyl or C₁—, C₂—, C₃— or C₄-heteroalkyl radical (for example, a group of formula —C(═N)NH₂ or —C(═N)CH₃).

Preference is furthermore given to compounds of formula (I) (wherein U=phenyl) or (II) wherein V is a bond and W is a phenylene group substituted by a group of formula SO₂NH₂ or SO₂alkyl in the position ortho to V.

Preference is moreover given to compounds of formula (I) wherein A and Ar¹ together are an indole group to which the group X is bonded preferably in the 6-position.

Preference is also given to compounds of formula (I) wherein n is 0, A and Ar¹ together are a 6-indolyl group, X is CONH, m is 0, Ar² is a phenyl radical, Y is CO and R³ is a heterocycloalkyl or heteroalkylcycloalkyl group (especially a group of formula —N(CH₂CH₂)₂CH—CH(CH₂CH₂)₂NMe); special preference is given in this case to the stereochemistry at the phenylglycine entity being (R).

Owing to their substitution, compounds of formula (I) or (II) contain one or more centres of chirality. The present invention therefore includes both all pure enantiomers and all pure diastereoisomers and also mixtures thereof in any mixing ratio. The present invention moreover also includes all cis/trans-isomers of the compounds of the general formula (I) or (II) and also mixtures thereof. The present invention moreover includes all tautomeric forms of the compounds of formula (I) or (II).

Examples of pharmacologically acceptable salts of compounds of formula (I) or (II) are salts of physiologically acceptable mineral acids, such as hydrochloric acid, sulphuric acid and phosphoric acid; or salts of organic acids, such as methanesulphonic acid, p-toluenesulphonic acid, lactic acid, formic acid, acetic acid, trifluoro-acetic acid, citric acid, succinic acid, fumaric acid, maleic acid and salicylic acid. Compounds of formula (I) or (II) can be solvated, especially hydrated. The hydration may take place, for example, during the preparation process or as a consequence of the hygroscopic nature of the initially anhydrous compounds of formula (I) or (II).

The pharmaceutical compositions according to the present invention comprise at least one compound of formula (I) or (II) as active ingredient and optionally carrier substances and/or adjuvants.

The pro-drugs (for example, B. R. B. Silverman, Medizinische Chemie, VCH Weinheim, 1995, Chapter 8, p. 361ff), to which the present invention also relates consist of a compound of formula (I) or (II) and at least one pharmacologically acceptable protecting group that is removed under physiological conditions, for example a hydroxy, alkoxy, aralkyloxy, acyl or acyloxy group, such as, for example, a methoxy, ethoxy, benzyloxy, acetyl or acetyloxy group.

The compounds of formulae (I) and (II) described herein can be prepared according to methods known per se. Compounds of formulae (I) and (II) according to the invention can be prepared, for example, by reaction of compounds of formulae (III) (where appropriate, in hydrochloride form or in the form of a similar salt), (IV) and (V) using a multi-component reaction (A. Dömling, I. Ugi, Angew. Chem. 2000, 112, 3300-3344), the radicals being defined as above. In the process, a compound of formula (III) is preferably dissolved together with a compound of formula (IV) especially in a suitable solvent (preferably a mixture of acetonitrile and water) and, where appropriate, stirred (preferably for 30 minutes at room temperature). A compound of formula (V) is then added and, where appropriate, further stirring is carried out (preferably for 15 hours at room temperature). The optionally present solvent is then removed preferably in vacuo. The compounds prepared in the process can be purified, for example, by means of HPLC and separated into the individual stereoisomers. Where appropriate, it may be preferred to carry out the reaction in the presence of a Lewis acid (for example, indium trichloride, boron trifluoride etherate, trimethyl aluminium, lithium chloride, aluminium trichloride, scandium triflate, zinc chloride, ytterbium triflate, magnesium triflate, magnesium bromide, zirconium chloride, titanium(IV) chloride or tin tetrachloride) or a Bronsted acid. In the case of the compounds, obtained in that manner it was found that both the compounds of formula (I) and (II) having an (R) configuration at the phenylglycine entity and also the corresponding (S)-configured compounds are very effective factor Xa inhibitors, the (S)-configured compounds having, when identically substituted, slightly better inhibitory properties. Preference is therefore given in accordance with the invention to compounds of formula (I) and (II) having an (S) configuration, whilst compounds having an (R) configuration, and also mixtures in any mixing ratio, also have very good inhibitory properties and this invention relates also thereto.

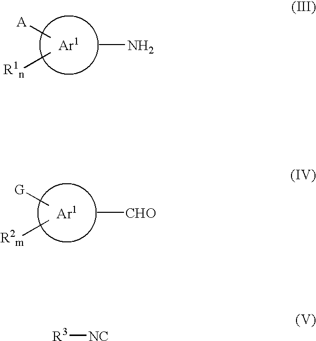

Alternatively, compounds of formula (I) or (II) can be prepared, for example, analogously to the methods described in WO0230880, WO02057236, WO0112600, WO0071493, WO0071508, WO0071507, WO0035858, WO02068390, WO0216312 and WO0190051.

3-Aminobenzamidine is commercially available; 3-amino-4-hydroxybenzamidine can be prepared from commercially available 4-hydroxy-3-nitrobenzonitrile by means of a Pinner reaction (A. Pinner, F. Klein, Ber. 10, 1889 (1877); 11, 4, 1475 (1878); 16, 352, 1643 (1883)) resulting in 4-hydroxy-3-nitro-benzamidine and subsequent reduction with H₂—Pd/C. Further benzamidines (such as, for example, 3-amino-4-chloro-benzamidine) can also be prepared analogously.

Glycosylated aryl compounds (for example, glycosylated benzaldehydes) can be prepared, for example, by the processes described in Kleine et al. Carbohydrate Research 1985, 142, 333-337 and Brewster et al. Tetrahedron Letters 1979, 5051-5054.

Helicin (salicylaldehyde-β-D-glucoside) is commercially available.

A compound or pharmaceutical composition of the present invention can be used in inhibiting factor Xa activity, in the prevention and/or treatment of thromboembolic conditions, arterial restenosis, septicaemia, cancer, acute inflammation or other conditions mediated by factor Xₐ activity, and especially venous thromboses, oedema or inflammation, deep vein thrombosis, pulmonary embolisms, thromboembolic complications after relatively major operations, in the case of vascular surgery, prolonged immobilisation, fractures of the lower extremities etc., arterial thromboses, especially of the coronary vessels in the event of myocardial infarct, and arteriosclerosis, stroke, angina pectoris, intermittent claudication, to, mention but a few indications.

In general, as mentioned at the beginning, the active ingredients according to the invention are to have an inhibitory action towards factor Xa that is as great as possible while having a selectivity that is as high as possible. The selectivity was assessed in the present case by comparing the inhibitory action towards factor Xa and also tryptase, trypsin, plasmin, thrombin and further serine proteases. Furthermore, the present compounds according to-the invention are of interest as inhibitors of further enzymes of the coagulation cascade (extrinsic and intrinsic) such as, for example, factor II, factor VII, factor VIIa, factor IX, factor IXa and factor X.

As mentioned above, the therapeutic use of the compounds of formula (I) or (II), of their pharmacologically acceptable salts and solvates and hydrates and also formulations and pharmaceutical compositions lies within the scope of the present invention.

The present invention relates also to the use of those active ingredients in the preparation of medicaments for the prevention and/or treatment of thromboembolic conditions. In general, compounds of formula (I) or (II) are administered either individually or in combination with any other desired therapeutic agent, using the known and acceptable methods. Administration may be effected, for example, by one of the following routes: orally, for example in the form of dragees, coated tablets, pills, semi-solid substances, soft or hard capsules, solutions, emulsions or suspensions; parenterally, for example in the form of an injectable solution; rectally in the form of suppositories; by inhalation, for example in the form of a powder formulation or spray, transdermally or intranasally. For the preparation of such tablets, pills, semi-solid substances, coated tablets, dragees and hard gelatin capsules, the therapeutically usable product can be mixed with pharmacologically inert, inorganic or organic pharmaceutical carrier substances, for example with lactose, sucrose, glucose, gelatin, malt, silica gel, starch or derivatives thereof, talcum, stearic acid or salts thereof, skimmed milk powder and the like. For the preparation of soft capsules, pharmaceutical carrier substances such as, for example, vegetable oils, petroleum, animal or synthetic oils, wax, fat and polyols can be used. For the preparation of liquid solutions and syrups, pharmaceutical carrier substances such as, for example, water, alcohols, aqueous saline solution, aqueous dextrose, polyols, glycerol, vegetable oils, petroleum and animal or synthetic oils can be used. For suppositories, pharmaceutical carrier substances such as, for example, vegetable oils, petroleum, animal or synthetic oils, wax, fat and polyols can be used. For aerosol formulations, compressed gases that are suitable for the purpose can be used, such as, for example, oxygen, nitrogen and carbon dioxide. The pharmaceutically acceptable agents may also comprise additives for preserving and stabilising, emulsifiers, sweeteners, flavourings, salts for altering the osmotic pressure, buffers, encapsulation additives and anti-oxidants.

Combinations with other therapeutic agents may comprise other active ingredients that are customarily used for the prevention and/or treatment of thromboembolic conditions, such as, for example, warfarin etc.

For the prevention and/or treatment of the conditions mentioned above, the dose of the biologically active compound according to the invention can vary within wide limits and can be adjusted to individual requirements. In general, a dose of from 0.1 μg to 10 mg/kg of body weight per day is suitable, a preferred dose being from 0.1 to 4 mg/kg per day. In suitable cases, the dose may also be below or above the stated values.

The daily dose can be administered in, for example, 1, 2, 3 or 4 individual doses. It is also possible to administer the dose as a single dose for, for example, one week.

The following Examples are intended to illustrate the invention. The stereochemistry of 3,4,5-trihydroxy-6-hydroxymethyl-tetrahydropyran-2-yloxy corresponds to that of β-D-glucose, and that of Examples 58, 59 and 60 corresponds to that of β-D-galactose.

### EXAMPLES

General procedure:

1 mmol of amine (II) and 1 mmol of aldehyde (III) are stirred in 20 ml of acetonitrile/water (mixing ratio of from 1:0 to 1:1) for 30 minutes at room temperature. 1 mmol of isonitrile (IV) is then added and stirring is carried out for a further 15 hours. The solvent is removed in vacuo and the residue is purified by means of HPLC.

### Example 1

### 2-(3-Carbamimidoyl-phenylamino)-N-(2-trifluoromethyl-benzyl)-2-[2-(3,4,5-trihydroxy-6-hydroxymethyl-tetrahydro-pyran-2-yloxy)-phenyl]-acetamide

C₂₉H₃₁F₃N₄O₇ (604.5882) ESI-TOF MS: 605 [M+H]

### Example 2

### 2-(3-Carbamimidoyl-phenylamino)-N-(2,3-dihydro-benzo[1,4]dioxin-6-yl)-2-[2-(3,4,5-trihydroxy-6-hydroxymethyl-tetrahydro-pyran-2-yloxy)-phenyl]-acetamide

C₂₉H₃₂N₄O₉ (580.5998) ESI-TOF MS: 581 [M+H]

### Example 3

### 2-(3-Carbamimidoyl-phenylamino)-N-[3-(2-oxo-pyrrolidin-1-yl)-propyl]-2-[2-(3,4,5-trihydroxy-6-hydroxymethyl-tetrahydro-pyran-2-yloxy)-phenyl]-acetamide

C₂₈H₃₇N₅O₈ (571.6358) ESI-TOF MS: 572 [M+H]

### Example 4

### 2-(3-Carbamimidoyl-phenylamino)-N-(4-phenoxy-phenyl)-2-[2-(3,4,5-trihydroxy-6-hydroxymethyl-tetrahydro-pyran-2-yloxy)-phenyl3-acetamide

C₃₃H₃₄N₄O₈ (614.6609) ESI-TOF MS: 615 [M+H]

### Example 5

### 2-(3-Carbamimidoyl-phenylamino)-N-(3,3-diphenyl-propyl)-2-[2-(3,4,5-trihydroxy-6-hydroxymethyl-tetrahydro-pyran-2-yloxy)-phenyl]-acetamide

C₃₆H₄₀N₄O₇ (640.7428) ESI-TOF MS: 641 [M+H]

### Example 6

### 2-(3-Carbamimidoyl-phenylamino)-N-(3-phenoxy-phenyl)-2-[2-(3,4,5-trihydroxy-6-hydroxymethyl-tetrahydro-pyran-2-yloxy)-phenyl]-acetamide

C₃₃H₃₄N₄O₈ (614.6609) ESI-TOF MS: 615 [M+H]

### Example 7

### 2-(3-Carbamimidoyl-phenylamino)-N-(4-methoxy-biphenyl-3-yl)-2-[2-(3,4,5-trihydroxy-6-hydroxymethyl-tetrahydro-pyran-2-yloxy)-phenyl]-acetamide

C₃₄H₃₆N₄O₈ (628.6880) ESI-TOF MS: 629 [M+H]

### Example 8

### 2-(3-Carbamimidoyl-phenylamino)-N-(4-morpholin-4-yl-phenyl)-2-[2-(3,4,5-trihydroxy-6-hydroxymethyl-tetrahydro-pyran-2-yloxy)-phenyl]-acetamide

C₃₁H₃₇N₅O₈ (607.6692) ESI-TOF MS: 608 [M+H]

### Example 9

### 2-(3-Carbamimidoyl-phenylamino)-N-(4-benzoyl-phenyl)-2-[2-(3,4,5-trihydroxy-6-hydroxymethyl-tetrahydro-pyran-2-yloxy)-phenyl]-acetamide

C₃₄H₃₄N₄O₈ (626.6721) ESI-TOF MS: 627 [M+H]

### Example 10

### 2-(3-Carbamimidoyl-phenylamino)-N-(3-benzoyl-phenyl)-2-[2-(3,4,5-trihydroxy-6-hydroxymethyl-tetrahydro-pyran-2-yloxy)-phenyl]-acetamide

C₃₄H₃₄N₄O₈ (626.6721) ESI-TOF MS: 627 [M+H]

### Example 11

### 2-(3-Carbamimidoyl-phenylamino)-N-(4-tert-butyl-benzyl)-2-[2-(3,4,5-trihydroxy-6-hydroxymethyl-tetrahydro-pyran-2-yloxy)-phenyl]-acetamide

C₃₂H₄₀N₄O₇ (592.6982) ESI-TOF MS: 593 [M+H]

### Example 12

### 2-(2-Hydroxy-5-carbamimidoyl-phenylamino)-N-(4-morpholin-4-yl-phenyl)-2-[2-(3,4,5-trihydroxy-6-hydroxymethyl-tetrahydro-pyran-2-yloxy)-phenyl]-acetamide

C₃₁H₃₇N₅O₉ (623.6686) ESI-TOF MS: 624 [M+H]

### Example 13

### 2-(3-Carbamimidoyl-phenylamino)-N-(3-methoxy-benzyl)-2-[2-(3,4,5-trihydroxy-6-hydroxymethyl-tetrahydro-pyran-2-yloxy)-phenyl]-acetamide

C₂₉H₃₄N₄O₈ (566.6163) ESI-TOF MS: 567 [M+H]

### Example 14

### N-(4-Acetyl-phenyl)-2-(3-carbamimidoyl-phenylamino)-2-[2-(3,4,5-trihydroxy-6-hydroxymethyl-tetrahydro-pyran-2-yloxy)-phenyl]-acetamide

C₂₉H₃₂N₄O₈ (564.6004) ESI-TOF MS: 565 [M+H]

### Example 15

### 2-(3-Carbamimidoyl-phenylamino)-N-(3-trifluoromethyl-benzyl)-2-[2-(3,4,5-trihydroxy-6-hydroxymethyl-tetrahydro-pyran-2-yloxy)-phenyl]-acetamide

C₂₉H₃₁F₃N₄O₇ (604.5882) ESI-TOF MS: 605 [M+H]

### Example 16

### 2-(3-Carybamimidoyl-phenylamino)-N-(2-cyclohex-1-enyl-ethyl)-2-[2-(3,4,5-trihydroxy-6-hydroxymethyl-tetrahydro-pyran-2-yloxy)-phenyl]-acetamide

C₂₉H₃₈N₄O₇ (554.6488) ESI-TOF MS: 555 [M+H]

### Example 17

### 2-(3-Carbamimidoyl-phenylamino)-N-[2-(3,4-dimethoxy-phenyl)-ethyl]-2-[2-(3,4,5-trihydroxy-6-hydroxymethyl-tetrahydro-pyran-2-yloxy)-phenyl]-acetamide

C₃₁H₃₈N₄O₉ (610.6699) ESI-TOF MS: 611 [M+H]

### Example 18

### 2-(3-Carbamimidoyl-phenylamino)-N-(3-morpholin-4-yl-propyl)-2-[2-(3,4,5-trihydroxy-6-hydroxymethyl-tetrahydro-pyran-2-yloxy)-phenyl]-acetamide

C₂₈H₃₉N₅O₈ (573.6517) ESI-TOF MS: 574 [M+H]

### Example 19

### 2-(3-Carbamimidoyl-phenylamino)-N-(4-trifluoromethyl-benzyl)-2-[2-(3,4,5-trihydroxy-6-hydroxymethyl-tetrahydro-pyran-2-yloxy)-phenyl]-acetamide

C₂₉H₃₁F₃N₄O₇ (604.5882) ESI-TOF MS: 605 [M+H]

### Example 20

### N-[1-(4-Bromo-phenyl)-ethyl]-2-(3-carbamimidoyl-phenylamino)-2-[2-(3,4,5-trihydroxy-6-hydroxymethyl-tetrahydro-pyran-2-yloxy)-phenyl]-acetamide

C₂₉H₃₃BrN₄O₇ (629.5130) ESI-TOF MS: 630 [M+H]

### Example 21

### N-Benzo[1,3]dioxol-5-ylmethyl-2-(3-carbamimidoyl-phenylamino)-2-[2-(3,4,5-trihydroxy-6-hydroxymethyl-tetrahydro-pyran-2-yloxy)-phenyl]-acetamide

C₂₉H₃₂N₄O₉ (580.5998) ESI-TOF MS: 581 [M+H]

### Example 22

### 2-(3-Carbamimidoyl-phenylamino)-N-(3-phenyl-propyl)-2-[2-(3,4,5-trihydroxy-6-hydroxymethyl-tetrahydro-pyran-2-yloxy)-phenyl]-acetamide

C₃₀H₃₆N₄O₇ (564.6440) ESI-TOF MS: 565 [M+H]

### Example 23

### 2-(3-Carbamimidoyl-phenylamino)-N-(3,5-dimethyl-benzyl)-2-[2-(3,4,5-trihydroxy-6-hydroxymethyl-tetrahydro-pyran-2-yloxy)-phenyl]-acetamide

C₃₀H₃₆N₄O₇ (564.6440) ESI-TOF MS: 565 [M+H]

### Example 24

### 2-(3-Carbamimidoyl-phenylamino)-N-(3-cyano-phenyl)-2-[2-(3,4,5-trihydroxy-6-hydroxymethyl-tetrahydro-pyran-2-yloxy)-phenyl]-acetamide

C₂₈H₂₉N₅O₇ (547.5726) ESI-TOF MS: 548 [M+H]

### Example 25

### 2-(3-Carbamimidoyl-phenylamino)-N-(3,4-dichloro-benzyl)-2-[2-(3,4,5-trihydroxy-6-hydroxymethyl-tetrahydro-pyran-2-yloxy)-phenyl]-acetamide

C₂₈H₃₀C₁₂N₄O₇ (605.4799) ESI-TOF MS: 606 [M+H]

### Example 26

### N-(3-Acetyl-phenyl)-2-(3-carbamimidoyl-phenylamino)-2-[2-(3,4,5-trihydroxy-6-hydroxymethyl-tetrahydro-pyran-2-yloxy)-phenyl]-acetamide

C₂₉H₃₂N₄O₈ (564.6004) ESI-TOF MS: 565 [M+H]

### Example 27

### 2-(3-Carbamimidoyl-phenylamino)-2-[2-(3,4,5-trihydroxy-6-hydroxymethyl-tetrahydro-pyran-2-yloxy)-phenyl]-N-(1,2,2-trimethyl-propyl)-acetamide

C₂₇H₃₈N₄O₇ (530.6265) ESI-TOF MS: 531 [M+H]

### Example 28

### N-Allyl-2-(3-carbamimidoyl-phenylamino)-2-[2-(3,4,5-trihydroxy-6-hydroxymethyl-tetrahydro-pyran-2-yloxy)-phenyl]-acetamide

C₂₄H₃₀N₄O₇ (486.5293) ESI-TOF MS: 487 [M+H]

### Example 29

### N-(3-Butoxy-propyl)-2-(3-carbamimidoyl-phenylamino)-2-[2-(3,4,5-trihydroxy-6-hydroxymethyl-tetrahydro-pyran-2-yloxy)-phenyl]-acetamide

C₂₈H₄₀N₄O₈ (560.6530) ESI-TOF MS: 561 [M+H]

### Example 30

### 2-(3-Carbamimidoyl-phenylamino)-N-(3,7-dimethyl-octa-2,6-dienyl)-2-[2-(3,4,5-trihydroxy-6-hydroxymethyl-tetrahydro-pyran-2-yloxy)-phenyl]-acetamide

C₃₁H₄₂N₄O₇ (582.7030) ESI-TOF MS: 583 [M+H]

### Example 31

### 2-(3-Carbamimidoyl-phenylamino)-N-furan-2-ylmethyl-2-[2-(3,4,5-trihydroxy-6-hydroxymethyl-tetrahydro-pyran-2-yloxy)-phenyl]-acetamide

C₂₆H₃₀N₄O₈ (526.5510) ESI-TOF MS: 527 [M+H]

### Example 32

### 2-(3-Carbamimidoyl-phenylamino)-N-(3-isopropoxy-propyl)-2-[2-(3,4,5-trihydroxy-6-hydroxymethyl-tetrahydro-pyran-2-yloxy)-phenyl]-acetamide

C₂₇H₃₈N₄O₈ (546.6259) ESI-TOF MS: 547 [M+H]

### Example 33

### 3-{2-(3-Carbamimidoyl-phenylamino)-2-[2-(3,4,5-trihydroxy-6-hydroxymethyl-tetrahydro-pyran-2-yloxy)-phenyl]-acetylamino}-propionic acid ethyl ester

C₂₆H₃₄N₄O₉ (546.5823) ESI-TOF MS: 547 [M+H]

### Example 34

### N-tert-Butyl-2-(3-carbamimidoyl-phenylamino)-2-[2-(3,4,5-trihydroxy-6-hydroxymethyl-tetrahydro-pyran-2-yloxy)-phenyl]-acetamide

C₂₅H₃₄N₄O₇ (502.5723) ESI-TOF MS: 503 [M+H]

### Example 35

### 2-(3-Carbamimidoyl-phenylamino)-N-pyridin-4-ylmethyl-2-[2-(3,4,5-trihydroxy-6-hydroxymethyl-tetrahydro-pyran-2-yloxy)-phenyl]-acetamide

C₂₇H₃₁N₅O₇ (537.5774) ESI-TOF MS: 538 [M+H]

### Example 36

### 2-(3-Carbamimidoyl-phenylamino)-N-methyl-2-[2-(3,4,5-trihydroxy-6-hydroxymethyl-tetrahydro-pyran-2-yloxy)-phenyl]-acetamide

C₂₂H₂₈N₄O₇ (460.4911) ESI-TOF MS: 461 [M+H]

### Example 37

### 2-(3-Carbamimidoyl-phenylamino)-N-(1,3-dimethyl-butyl)-2-[2-(3,4,5-trihydroxy-6-hydroxymethyl-tetrahydro-pyran-2-yloxy)-phenyl]-acetamide

C₂₇H₃₈N₄O₇ (530.6265) ESI-TOF MS: 531 [M+H]

### Example 38

### N-(4-Benzoyl-phenyl)-2-(3-carbamimidoyl-phenylamino)-2-[2-(3,4,5-trihydroxy-6-hydroxymethyl-tetrahydro-pyran-2-yloxy)-phenyl]-acetamide

C₃₄H₃₄N₄O₈ (626.6721) ESI-TOF MS: 627 [M+H]

### Example 39

### 2-(5-Carbamimidoyl-2-hydroxy-phenylamino)-N-(4-morpholin-4-yl-phenyl)-2-[2-(3,4,5-trihydroxy-6-hydroxy-methyl-tetrahydro-pyran-2-yloxy)-phenyl]-acetamide

C₃₁H₃₇N₅O₉ (623.6686) ESI-TOF MS: 624 [M+H]

### Example 40

### 2-(3-Carbamimidoyl-phenylamino)-N-(2,6-dimethyl-phenyl)-2-[2-(3,4,5-trihydroxy-6-hydroxymethyl-tetrahydro-pyran-2-yloxy)-phenyl]-acetamide

C₂₉H₃₄N₄O₇ (550.6169) ESI-TOF MS: 551 [M+H]

### Example 41

### 2-(3-Carbamimidoyl-phenylamino)-N-(4-nitro-phenyl)-2-[2-(3,4,5-trihydroxy-6-hydroxymethyl-tetrahydro-pyran-2-yloxy)-phenyl]-acetamide

C27H29N5O9 (567.5603) ESI-TOF MS: 568 [M+H]

### Example 42

### N-(4-Amino-phenyl)-2-(3-carbamimidoyl-phenylamino)-2-[2-(3,4,5-trihydroxy-6-hydroxymethyl-tetrahydro-pyran-2-yloxy)-phenyl]-acetamide

C27H31N5O7 (537.5774) ESI-TOF MS: 538 [M+H]

### Example 43

### 2-{2-(3-Carbamimidoyl-phenylamino)-2-[2-(3,4,5-trihydroxy-6-hydroxymethyl-tetrahydro-pyran-2-yloxy)-phenyl]-acetylamino}-benzoic acid methyl ester

C₂₉H₃₂N₄O₉ (580.5998) ESI-TOF MS: 581 [M+H]

### Example 44

### 2-(5-Carbamimidoyl-2-chloro-phenylamino)-N-(4-morpholin-4-yl-phenyl)-2-[2-(3,4,5-trihydroxy-6-hydroxy-methyl-tetrahydro-pyran-2-yloxy)-phenyl]-acetamide

C₃₁H₃₆ClN₅O₈ (642.1143) ESI-TOF MS: 643 [M+H]

### Example 45

### 2-(3-Carbamimidoyl-phenylamino)-N-[4-(morpholin-4-carbonyl)-phenyl]-2-[2-(3,4,5-trihydroxy-6-hydroxymethyl-tetrahydro-pyran-2-yloxy)-phenyl]-acetamide

C₃₂H₃₇N₅O₉ (635.6798) ESI-TOF MS: 636 [M+H]

### Example 46

### 2-(5-Carbamimidoyl-2-methylamino-phenylamino)-N-(4-morpholin-4-yl-phenyl)-2-[2-(3,4,5-trihydroxy-6-hydroxymethyl-tetrahydro-pyran-2-yloxy)-phenyl]-acetamide

C₃₂H₄₀N₆O₈ (636.7110) ESI-TOF MS: 637 [M+H]

### Example 47

### N-(2-Bromo-phenyl)-2-(3-carbamimidoyl-phenyl-amino)-2-[2-(3,4,5-trihydroxy-6-hydroxymethyl-tetrahydro-pyran-2-yloxy)-phenyl]-acetamide

C₂₇H₂₉BrN₄O₇ (601.4588) ESI-TOF MS: 602 [M+H]

### Example 48

### 2-{2-(3-Carbamimidoyl-phenylamino)-2-[2-(3,4,5-trihydroxy-6-hydroxymethyl-tetrahydro-pyran-2-yloxy)-phenyl]-acetylamino}-benzoic acid

C₂₈H₃₀N₄O₉ (566.5727) ESI-TOF MS: 567 [M+H]

### Example 49

### 2-(3-Carbamimidoyl-phenylamino)-N-quinolin-6-yl-2-[2-(3,4,5-trihydroxy-6-hydroxymethyl-tetrahydro-pyran-2-yloxy)-phenyl]-acetamide

C₃₀H₃₁N₅O₇ (573.6109) ESI-TOF MS: 574 [M+H]

### Example 50

### 2-{2-{3-[N-(3-Fluoro-benzyl)-carbamimidoyl]-phenylamino}-2-[2-(3,4,5-trihydroxy-6-hydroxymethyl-tetrahydro-pyran-2-yloxy)-phenyl]-acetylamino}-benzoic acid methyl ester

C₃₆H₃₇FN₄O₉ (688.7161) ESI-TOF MS: 689 [M+H]

### Example 51

### N-(2-Benzoyl-4-chloro-phenyl)-2-(3-carbamimidoyl-phenylamino)-2-[2-(3,4,5-trihydroxy-6-hydroxymethyl-tetrahydro-pyran-2-yloxy)-phenyl]-acetamide

C₃₄H₃₃ClN₄O₈ (661.1171) ESI-TOF MS: 662 [M+H]

### Example 52

### 2-(3-Carbamimidoyl-phenylamino)-N-[3-chloro-4-(morpholin-4-carbonyl)-phenyl]-2-[2-(3,4,5-trihydroxy-6-hydroxymethyl-tetrahydro-pyran-2-yloxy)-phenyl]-acetamide

C₃₂H₃₆ClN₅O₉ (670.1248) ESI-TOF MS: 671 [M+H]

### Example 53

### 2-(3-Carbamimidoyl-phenylamino)-N-[2-methyl-4-(morpholin-4-carbonyl)-phenyl]-2-[2-(3,4,5-trihydroxy-6-hydroxymethyl-tetrahydro-pyran-2-yloxy)-phenyl]-acetamide

C₃₃H₃₉N₅O₉ (649.7069) ESI-TOF MS: 650 [M+H]

### Example 54

### 2-(5-Carbamimidoyl-2-hydroxy-phenylamino)-N-[4-(morpholin-4-carbonyl)-phenyl]-2-[2-(3,4,5-trihydroxy-6-hydroxymethyl-tetrahydro-pyran-2-yloxy)-phenyl]-acetamide

C₃₂H₃₇N₅O₁₀ (651.6792) ESI-TOF MS: 652 [M+H]

### Example 55

### 2-(3-Carbamimidoyl-phenylamino)-N-(3,4-difluoro-phenyl)-2-[2-(3,4,5-trihydroxy-6-hydroxymethyl-tetrahydro-pyran-2-yloxy)-phenyl]-acetamide

C₂₇H₂₈F₂N₄O₇ (558.5436) ESI-TOF MS: 559 [M+H]

### Example 56

### 4-{2-(3-Carbamimidoyl-phenylamino)-2-[2-(3,4,5-trihydroxy-6-hydroxymethyl-tetrahydro-pyran-2-yloxy)-phenyl]-acetylamino}-benzoic acid methyl ester

C₂₉H₃₂N₄O₉ (580.5998) ESI-TOF MS: 581 [M+H]

### Example 57

### 2-(3-Carbamimidoyl-phenylamino)-N-(3,4-dimethoxy-phenyl)-2-[2-(3,4,5-trihydroxy-6-hydroxymethyl-tetrahydro-pyran-2-yloxy)-phenyl]-acetamide

C₂₉H₃₄N₄O₉ (582.6157) ESI-TOF MS: 583 [M+H]

### Example 58

### Acetic acid 3,4,5-triacetoxy-6-{2-[(5-carbamimidoyl-2-hydroxy-phenylamino)-(4-morpholin-4-yl-phenylcarbamoyl)-methyl]-phenoxy}-tetrahydro-pyran-2-ylmethyl ester

C₃₉H₄₅N₅O₁₃ (791.8192) ESI-TOF MS: 792 [M+H]

### Example 59

### 2-(3-Carbamimidoyl-phenylamino)-N-(4-morpholin-4-yl-phenyl)-2-[2-(3,4,5-trihydroxy-6-hydroxy-methyl-tetrahydro-pyran-2-yloxy)-phenyl]-acetamide

C₃₁H₃₇N₅O₈ (607.6692) ESI-TOF MS: 608 [M+H]

### Example 60

### 2-(5-Carbamimidoyl-2-hydroxy-phenylamino)-N-(4-morpholin-4-yl-phenyl)-2-[2-(3,4,5-trihydroxy-6-hydroxymethyl-tetrahydro-pyran-2-yloxy)-phenyl]-acetamide

C₃₁H₃7N₅O₉ (623.6686) ESI-TOF MS: 624 [M+H]

### Example 61

### 2-(3-Carbamimidoyl-phenylamino)-N-(4-methoxy-phenyl)-2-[2-(3,4,5-trihydroxy-6-hydroxymethyl-tetrahydro-pyran-2-yloxy)-phenyl]-acetamide

C₂₈H₃₂N₄O₈ (552.5892) ESI-TOF MS: 553 [M+H]

### Example 62

### 2-(3-Carbamimidoyl-phenylamino)-2-[3-ethoxy-2-(3,4,5-trihydroxy-6-hydroxymethyl-tetrahydro-pyran-2-yloxy)-phenyl]-N-[4-(morpholine-4-carbonyl)-phenyl]-acetamide

C₃₄H₄₁N₅O₁₀ (679.7334) ESI-TOF MS: 680 [M+H]

### Example 63

### 2-(3-Carbamimidoyl-phenylamino)-2-[3-ethoxy-2-(3,4,5-trihydroxy-6-hydroxymethyl-tetrahydro-pyran-2-yloxy)-phenyl]-N-(4-morpholin-4-yl-phenyl)-acetamide

C₃₃H₄₁N₅O₉ (651.7228) ESI-TOF MS: 652 [M+H]

In order to demonstrate the inhibitory action towards factor Xa activity, chromogenic peptide substrates were used. The inhibition of the amidolytic activity of factor Xa by the compounds described above was demonstrated as follows. The measurements were carried out in microtitre plates at room temperature. The compounds were dissolved in dimethyl sulphoxide and 5 μl of the solution (1 mM, 100 μM, 10 μM, 1 μM) were added to 35 μml of a 2.15 nM solution of human recombinant factor Xa (Enzyme Research Laboratories, South Bend, Ind., USA) in a buffer (pH: 8.0 and using 50 mM Tris-HCl, 100 mM NaCl, 0.1% PEG 6000 and 0.05% Tween 80). Finally, 10 μl of a 25 μM MeSO₂-D-CHA-Gly-Arg-AMC acetate solution (“Spectrozym fXa”, American Diagnostica, Pfungstadt, Germany) in buffer were added and the hydrolysis of the substrate was monitored with a Spectra Fluor Plus spectrophotometer (Tecan, Crailsheim, Germany) over a period of 20 minutes at the following wavelengths: excitation: 360 nm, emission: 465 nm. The IC₅₀ values were calculated by means of the “GraFit 4” program of the company Erithacus Software Ltd. (Staines, Middlesex, UK). On the assumption that the kinetics comprise a competitive inhibition, it was possible to determine the Ki value by the Cheng-Prusoff equation: Ki=IC₅₀/(1+[S]/Kₘ]) (Cheng and Prusoff, Biochemical Pharmacology 1973, 22: 3099-3108). The same procedure, but with tosyl-glycyl-prolyl-lysine-4-nitranilide acetate being used as the substrate in Hepes buffer (pH 7.8), was used to determine the inhibition of the proteolytic activity of recombinant human tryptase (Promega, Madison, Wis., USA) by the said compounds.

The IC₅₀ values of the above-mentioned Examples are in the range from 0.1 nM to 1 μM.

1. A compound comprising formula (I): 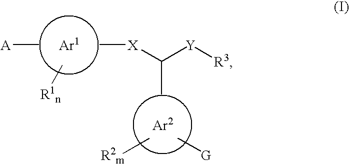
wherein
A is a hydrogen atom; a group of formula —C(═NR⁴)NH₂, wherein R⁴ is a hydrogen atom, a heteroalkyl, hetero-aralkyl, heterocycloalkyl, heteroalkylcycloalkyl, hydroxy or alkyloxy group or is, together with one of the radicals R¹, part of a 5- or 6-membered heteroaryl or heterocycloalkyl ring; a group of formula —NHC(═NR⁴)NH₂; or has one of the following structures: 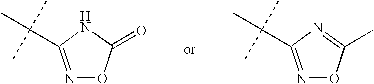
Ar¹ is an aryl, aralkyl, heteroaryl or heteroaralkyl group,
Ar² is an aryl, aralkyl, heteroaryl or heteroaralkyl group,
the radicals R¹ are, each independently of any other(s), a hydroxy group, a C₁-C₄alkyloxy group, an amino group, a C₁-C₄alkylamino group, a C₁-C₄-dialkylamino group, a cyano group or a halogen atom;
the radicals R², each independently of any other(s), are a hydroxy group, a C₁-C₄alkyloxy group, an amino group, a C₁-C₄alkylamino group, a C₁-C₄-dialkylamino group, a cyano group or a halogen atom;
R³ is an alkyl, alkenyl, alkynyl, heteroalkyl, aryl, heteroaryl, cycloalkyl, alkylcycloalkyl, heteroalkyl-cycloalkyl, heterocycloalkyl, aralkyl or heteroaralkyl radical;
G is a glycosyl group;
X is a group of formula NR⁵, O, CONR⁵, NR⁵CO, CH₂NR⁵, S, SO, SO₂, SO₂NH, NHSO₂, PO₂NH, NHPO₂, CH₂, CHMe or CO, wherein R⁵ is a hydrogen atom, a C₁-C₄alkyl, C₁-C₄-heteroalkyl, C₇-C₁₂aralkyl or C₆-C₁₂heteroaralkyl group;
Y is a group of formula CONR⁶, COCONR⁶, NR⁶, O, NR⁶CO, S, SO, SO₂, SO₂NH, NHSO₂, PO₂NH, NHPO₂, CH₂, CHMe or CO, wherein R⁶ is a hydrogen atom, a C₁-C₄alkyl, C₁-C₄-heteroalkyl or C₇-C₁₂aralkyl group;
n is 0, 1, 2, 3 or 4, and
m is 0, 1, 2, 3 or 4,
or a pharmacologically acceptable salt, solvate, hydrate or pharmacologically acceptable formulation thereof, there being excluded compounds in which Y is a group of formula CONR⁶ and R³ is a group of formula —CHR⁷—CO—NR⁸R⁹, R⁷, R⁸ and R⁹ being, each independently of the others, a hydrogen atom, an alkyl, alkenyl, alkynyl, heteroalkyl, heteroaralkyl, heteroaryl, aralkyl, cycloalkyl, heterocycloalkyl, alkylcyclo-alkyl, heteroalkylcycloalkyl or aryl group, or R⁸ and R⁹ together are part of a heterocycloalkyl or heteroaryl ring system; there furthermore being excluded compounds wherein Y is a group of formula CO and R3 is a group of formula —NR¹⁰—CHR⁷—CO—NR⁸R⁹, R⁷, R⁸, R⁹ and R¹⁰ being, each independently of the others, a hydrogen atom, an alkyl, alkenyl, alkynyl, heteroalkyl, heteroaralkyl, heteroaryl, alkylcyclo-alkyl, heteroalkyl-cycloalkyl, aralkyl, cycloalkyl, heterocycloalkyl or aryl group, or R⁸ and R⁹ and/or R⁷ and R¹⁰ together are part of a heterocycloalkyl or heteroaryl ring system.

2. Compounds according to claim 1, wherein A is a group of formula —C(═NH)NH₂.

3. Compounds according to claim 1, wherein Ar¹ is a phenyl or heteroaryl group having 5, 6, 7, 8, 9 or 10 carbon ring atoms and 1, 2, 3 or 4 ring hetero atoms selected from O, S and N.

4. Compounds according to claim 1, wherein Ar¹ is a phenyl group to which the groups A and X are bonded in positions meta to one another.

5. Compounds according to claim 1, wherein Ar² is a phenyl group.

6. Compounds according to claim 1, wherein X is an NH group.

7. Compounds according to claim 1, wherein n is 0 or 1.

8. Compounds according to claims 1, wherein R¹ is a hydroxy group.

9. Compounds according to claims 1, wherein m is 0 or 1.

10. Compounds according to claim 1, wherein Y is a group of formula CONH.

11. Compounds according to claims 1, wherein R³ is a group of formula —U—V—W, wherein U is an optionally substituted C₆-C₁₀aryl group or an optionally substituted hetero-aryl group containing from 5 to 10 ring atoms and 1, 2, 3 or 4 hetero atoms selected from O, S and N; V is a direct bond, an oxygen atom, a sulphur atom, a group of formula NR¹¹ (R¹¹ being a hydrogen atom, a C₁-C₄alkyl, C₁-C₄heteroalkyl, C₇-C₁₂aralkyl or C₆-C₁₂heteroaralkyl group), CO, SO, SO₂ or SO₂NH, and W is a hydrogen atom, an alkyl, alkenyl, alkynyl, heteroalkyl, aryl, heteroaryl, cycloalkyl, alkylcyclo-alkyl, aralkyl, heteroalkylcycloalkyl, heterocyclo-alkyl or heteroaralkyl radical.

12. Compounds according to claim 11, wherein U is an optionally substituted phenyl group.

13. Compounds according to claim 11, wherein V is a direct bond or a carbonyl group.

14. Compounds according to claim 11, wherein W is a C₁-C₄alkyl group, a C₁-C₄heteroalkyl group, an optionally substituted phenyl group, an optionally substituted C₃-C₇cycloalkyl group, an optionally substituted heterocycloalkyl group having 3-7 ring atoms and 1, 2 or 3 hetero atoms (selected from O, S and N) or an optionally substituted heteroaryl group having 5 or 6 ring atoms and 1, 2, 3 or 4 hetero atoms selected from O, S and N.

15. Pharmaceutical compositions comprising a compound according to claims 1, and, optionally, carrier substances and/or adjuvants.

16. A method for inhibiting factor Xa by administering a compound of claim 1.

17. A method for the treatment or prevention of thromboembolic conditions, arterial restenosis, septicaemia, cancer, acute inflammation or other conditions mediated by factor Xa activity comprising administering a compound of claim 1.

18. A method for preventing or reducing complications relating to thromboembolic conditions in vascular surgery comprising administering a compound of claim 1 prior to, during or following said surgery.
